(19) United States
(12) Patent Application Publication
(10) Pub. No.: US 2010/0191067 A1
(43) Pub. Date: Jul. 29, 2010

(54) Cervix Examination device and cervix examination set

(76) Inventor: Tien-Sheng Chen, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/499,127

(22) Filed: Jul. 8, 2009

(30) Foreign Application Priority Data
Jan. 23, 2009 (TW) 098201461

(51) Int. Cl. A61B 1/32

(52) U.S. Cl. 600/245

(57) ABSTRACT

A cervix examination device capable of containing and separating an inspection apparatus is disclosed. The cervix examination device comprises: a first blade; a second blade, which is pivoted to the first blade, comprising an open part and a holding part; an adjuster installed between the first and the second blades; and a container connected to the inner side of the second open part and the second holding part. The container comprises an open portion and a closed portion, and the open portion is located at the second holding part. At least a portion of the inspection apparatus can be contained in the closed portion via the open portion. The inspection apparatus comprises a light unit and a control unit. The control unit controls the light unit for the inspection process.

### BACKGROUND OF THE INVENTION

1. Field of the Invention

The present invention relates to a cervix examination device, capable of containing an inspection apparatus, and a cervix examination set, specifically to a cervix examination device and a cervix examination set suitable for cervical colposcopy to enable a user to distinguish abnormal cells from normal cells.

2. Description of the Related Art

In order to conduct PAPSURE™, a vaginal speculum needs to be inserted into the vagina to facilitate inspection or medication. In addition, during such an examination, a light source is needed to illuminate the inside of the vagina. Generally, the light source is usually a handheld light projector or a head-wearable light. However, the traditional light source is not convenient and cannot be inserted inside the vagina to provide sufficient illumination; thus, the illumination effect is poor when such traditional light sources are used.

To provide more desirable illumination means, several solutions have been proposed in prior arts. For example, a container is attached to the inner side of an open part of a vaginal speculum to contain and separate a light module. Because the light module is placed in the front of the vaginal speculum, the light module can provide illumination with higher efficiency inside the vagina. However, when a doctor uses the vaginal speculum, it is still possible that the light module could be contaminated because the open portion of the container is still close to the patient. Also, in prior arts, other auxiliary devices (e.g. a screen or a wireless transmitter) can not be added to the light module easily because of the structure layout.

Moreover, in prior arts, the doctor has to inspect by the naked eye without other auxiliary devices for saving, zooming, or analyzing the images of the inspection. This limits the effectiveness of PAPSURE™.

Therefore, it is desirable to provide a cervix examination device and a cervix examination set to mitigate and/or obviate the aforementioned problems.

### SUMMARY OF THE INVENTION

A primary object of the present invention is to provide a cervix examination device capable of containing and separating an inspection apparatus.

Another object is to provide a cervix examination set suitable for cervical colposcopy to enable a user to distinguish abnormal cells from normal cells.

In order to achieve the above-mentioned objects, the cervix examination set of the present invention comprises: a cervix examination device and an inspection apparatus. The cervix examination device comprises: a first blade comprising a first open part and a first holding part; a second blade pivoted to the first blade, the second blade comprising a second open part and a second holding part; an adjuster installed between the first blade and the second blade for adjusting the relative angle between the first and the second open parts; and a container connected to the inner side of the second open part and the second holding part. The container comprises an open portion and a closed portion. The open portion is located at the second holding part. At least a portion of the inspection apparatus can be contained in the closed portion via the open portion. The inspection apparatus comprises a light unit for emitting light; and a control unit. The control unit is electrically connected to the light unit and used for controlling the light unit.

According to one of the embodiments of the present invention, the inspection apparatus further comprises an image sensing unit, a wireless transmitter, a memory unit, and a display unit.

### BRIEF DESCRIPTION OF THE DRAWINGS

FIG. 1 is a schematic view of a cervix examination device of the present invention.

FIG. 2 is an exploded view of the cervix examination device of the present invention.

FIG. 3 is a schematic drawing of an inspection apparatus of a cervix examination set of the present invention.

FIG. 4 is a schematic drawing of the cervix examination set in accordance with one embodiment of the present invention.

FIG. 5 is a schematic drawing of the cervix examination set in accordance with an alternative embodiment of the present invention.

FIG. 6 is a schematic cross section view of the cervix examination set of the present invention when it is being used.

### DETAILED DESCRIPTION OF THE PREFERRED EMBODIMENT

The advantages and innovative features of the invention will become more apparent from the following detailed description when taken in conjunction with the accompanying drawings.

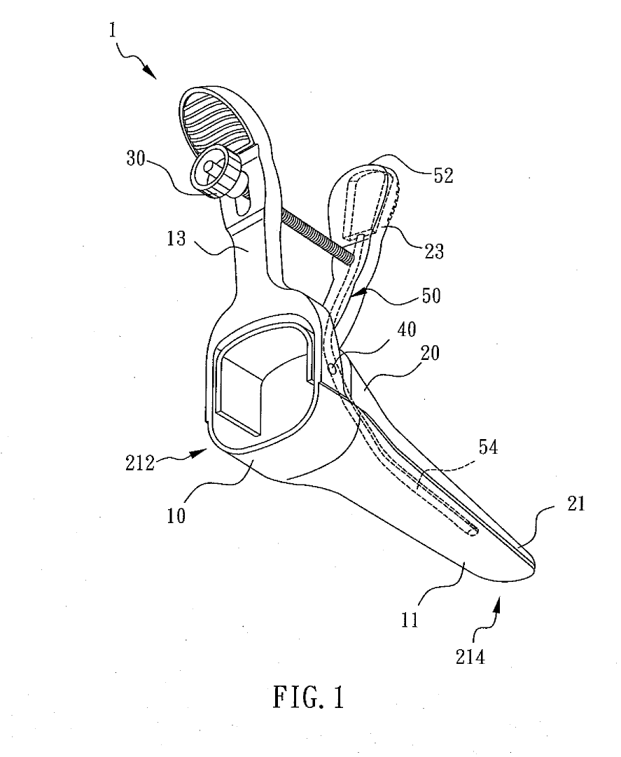

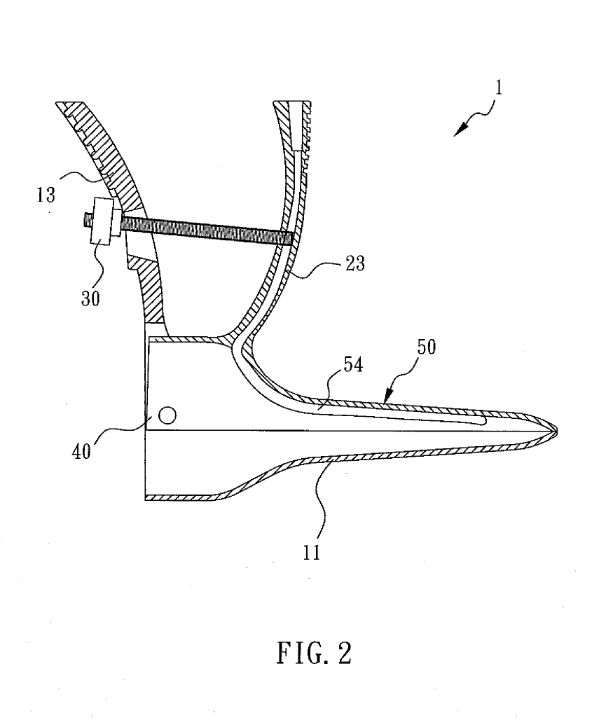

A cervix examination device 1 of the present invention may be applied to PAPSURE™, allowing a user to distinguish abnormal cells from normal cells, and ordinary cervical observations. Please refer to FIG. 1 and FIG. 2. The cervix examination device 1 mainly comprises a first blade 10, a second blade 20, an adjuster 30, and a container 50. The first blade 10 comprises a first open part 11 and a first holding part 13. The second blade 20 is pivoted to the first blade 10 by means of a pivot member 40. The second blade 20 comprises a second open part 21 and a second holding part 23. The adjuster 30 is installed between the first blade 10 and the second blade 20 for adjusting the relative angle between the first and the second open parts 11, 21. The container 50 comprises an open portion 52 and a closed portion 54. The container 50 is connected to the inner side of the second open part 21 and the second holding part 23, and the open portion 52 is located at the second holding part 23.

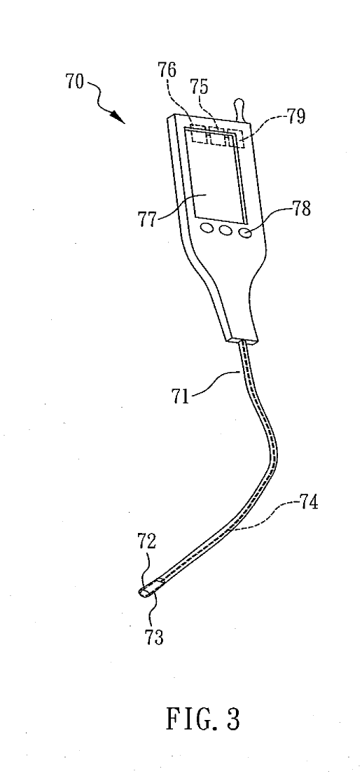

Next, please refer to FIG. 3. In this embodiment, an inspection apparatus 70 comprises a flexible tube 71, a light unit 72, an image sensing unit 73, a memory unit 75, a control unit 76, a screen 77, a switch 78, and a wireless transmitter 79. The control unit 76 is a control center of the inspection apparatus 70 and can be electrically coupled to the light unit 72, the image sensing unit 73, the memory unit 75, the screen 77, the switch 78, and the wireless transmitter 79. The user can conduct various desired operations with the switch 78. It should be noted that the components of the inspection apparatus 70 of the present invention are not limited to the above description. Some components of the inspection apparatus 70 can be removed, or additional components can be added to it, according to the user's requirements.

The light unit 72 is for emitting light. It may comprise a single color or multi-color LED (light emitting diode), or comprise a plurality of LEDs of different colors. However, the light unit 72 is not limited in above description. The light unit 72 also can be a fluorescent bar or other illumination objects. In this embodiment, the light unit 72 is electrically connected to the control unit 76 by a wire 74. The control unit 76 can then control the light unit 72 (such as to switch the light on/off and to change the color of the light emitted by the light unit).

In most situations, the light unit 72 may emit light of a color different from that of the normal cells, preferably green light, which is complementary to the red light reflected by normal cells, so that users may distinguish abnormal cells from normal cells more easily. However, the light unit 72 can also emit other visible light with wavelengths smaller than those of red light, for example and without limitation white light, yellow light, blue light, purple light, etc.

The image sensing unit 73 is for sensing images and can generate corresponding image data according to captured images and show image data on the screen 77 for analysis by the user. The screen 77 can be a liquid crystal display. The image sensing unit 73 can be a CCD (Charge Coupled Device), a CMOS (Complementary Metal Oxide Semiconductor) image sensing unit, or other type of image sensing unit.

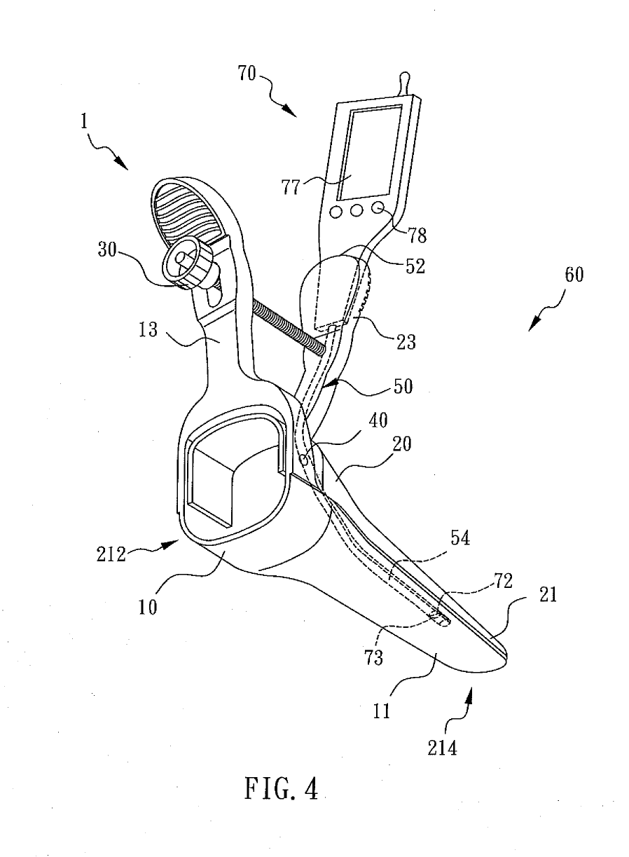

Please also refer to FIG. 4. The light unit 72 and the image sensing unit 73 are located at the end of the flexible tube 71 so that the user can put the flexible tube 71 together with the light unit 72, the image sensing unit 73, and the wire 74 into the closed portion 54 easily.

The wireless transmitter 79 can wirelessly transmit image data generated by the image sensing unit 73 to a remote computer (not shown in figures) for further analysis by and storage in the remote computer. Therefore, there is no need to use a physical wire to transmit image data to the remote computer, thus providing greater convenience. In addition, the image data generated by the image sensing unit 73 can also be saved in the memory unit 75 of the inspection apparatus 70.

As shown in FIG. 4, a cervix examination set 60 is a combination of the cervix examination device 1 and the inspection apparatus 70. At least a portion of the inspection apparatus 70 can be contained in the closed portion 54 via the open portion 52. The container 50 is used for containing and separating the inspection apparatus 70, whereby the inspection apparatus 70 will not be contaminated by contact with the patient. Therefore, the inspection apparatus 70 prevents cross infection and can be reused, thus achieving environmental protection and reducing the cost of the cervical examination. The closed portion 54 of the container 50 is in a slender form and extends toward the end 214 of the second open part 21, thereby allowing the inspection apparatus 70 to be used at deeper locations. The container 50 of the cervix examination device 1 of the cervix examination set 60 is connected to the inner side of the second open part 21 and the second holding part 23, and the open portion 52 is located at the end of the second holding part 23. Thus, the flexible tube 71 of the inspection apparatus 70 is contained in the closed portion 54 of the container 50. The other portion of the inspection apparatus 70 (i.e., the portion of the control panel) sticks out of the end of the second holding part 23 of the cervix examination device 1 so it will not obstruct a doctor's view when directly observing a cervix via an end 212. Moreover, a corresponding image (for example, zooming in a specific region of an image) can be displayed on the screen 77 simultaneously.

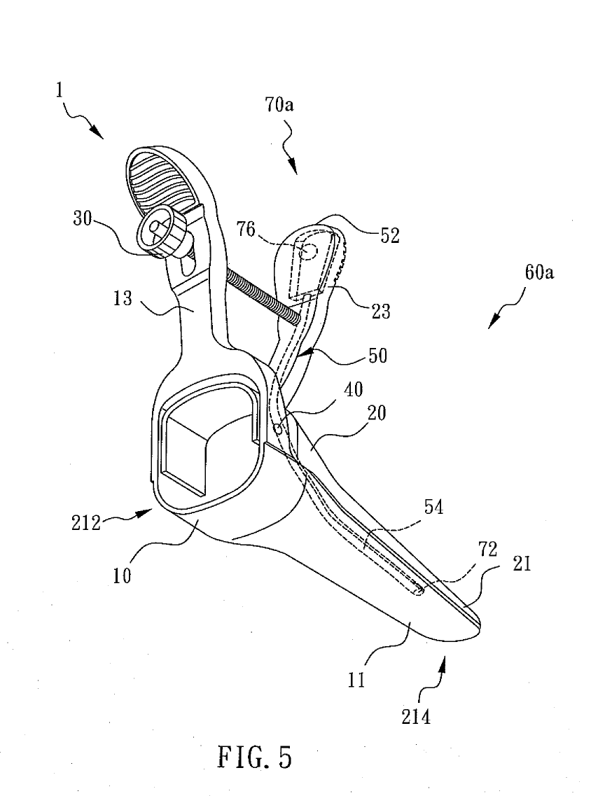

Next, please refer to FIG. 5, a schematic drawing of the cervix examination set in accordance with an alternative embodiment of the present invention. A cervix examination set 60a comprises the cervix examination device 1 and an inspection apparatus 70a. In this embodiment, the inspection apparatus 70a only has the function of emitting light and comprises the light unit 72 and the control unit 76. The light unit 72 is controlled by the control unit 76.

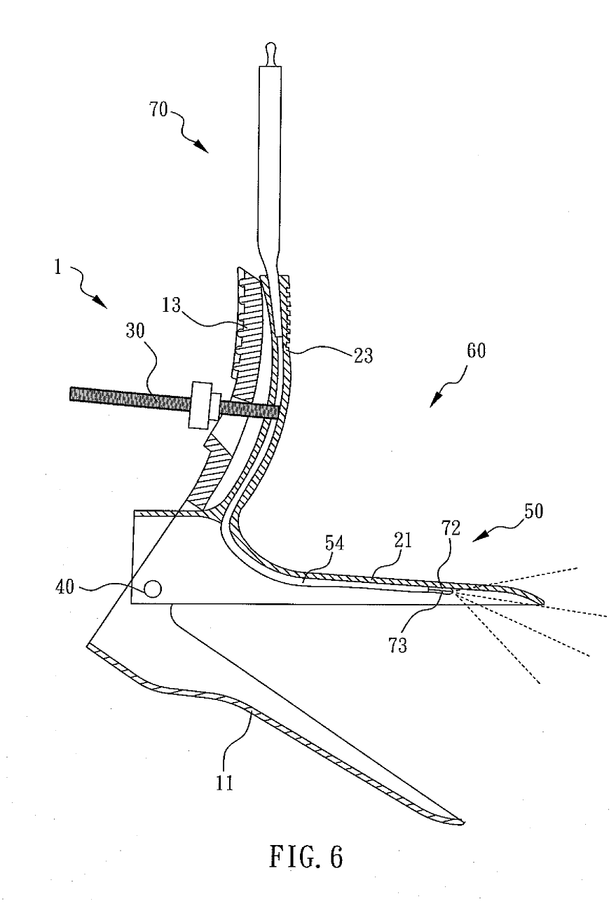

Please refer to FIG. 6 in conjunction with FIG. 3. FIG. 6 is a schematic cross section view of the cervix examination set of the present invention when it is being used. When the user operates the adjuster 30 to move the first holding part 13 closer to the second holding part 23, the angle between the first open part 11 and the second open part 21 will be increased. The flexible tube 71 of the inspection apparatus 70 is placed in the container 50, which is connected to the inner side of the second open part 21 and the second holding part 23. The flexible tube 71 is then substantially in an “L” shape in order to match the shape of the second open part 21 and the second holding part 23.

In conclusion, the cervix examination device 1 of the present invention is capable of containing and separating the flexible tube 71 of the inspection apparatus 70 by placing it in the container 50. The container 50 is connected to the inner side of the second open part 21 and the second holding part 23. The open portion 52 of the container 50 is located at the end of the second holding part 23, whereby the possibility that the open portion 52 may be in contact with the patient is reduced to a minimum, so the inspection apparatus 70 will not be contaminated. Moreover, the inspection apparatus 70 of the cervix examination set 60 of the present invention can further comprise such as the image sensing unit 73, the memory unit 75, the screen 77, the wireless transmitter 79, so as to further increase the correctness of and efficiency in the inspection.

It is noted that the above-mentioned embodiments are only for illustration. It is intended that the present description cover modifications and variations of this invention provided they fall within the scope of the following claims and their equivalents. Therefore, it will be apparent to those skilled in the art that various modifications and variations can be made to the structure of the present invention without departing from the scope or spirit of the invention.

What is claimed is:

1. A cervix examination device capable of containing and separating an inspection apparatus, the cervix examination device comprising:
a first blade comprising a first open part and a first holding part;
a second blade pivoted to the first blade, the second blade comprising a second open part and a second holding part;
an adjuster installed between the first blade and the second blades for adjusting a relative angle between the first and the second open parts; and
a container connected to the inner side of the second open part and the second holding part, the container comprising an open portion and a closed portion, the open portion being located at the second holding part, and at least a portion of the inspection apparatus can be contained in the closed portion via the open portion.

2. The cervix examination device as claimed in claim 1, wherein the closed portion of the container is in a slender form.

3. The cervix examination device as claimed in claim 1, wherein the closed portion of the container is substantially L-shaped.

4. A cervix examination set comprising:
a first blade comprising a first open part and a first holding part;
a second blade pivoted to the first blade, the second blade comprising a second open part and a second holding part;
an adjuster installed between the first blade and the second blade for adjusting a relative angle between the first and the second open parts;
an inspection apparatus comprising:
a light unit for emitting light; and
a control unit electrically connected to the light unit, the control unit being used for controlling the light unit; and
a container connected to the inner side of the second open part and the second holding part, the container comprising an open portion and a closed portion, the open portion being located at the second holding part, and at least a portion of the inspection apparatus can be contained in the closed portion via the open portion.

5. The cervix examination set as claimed in claim 4, wherein the control unit is for controlling color of the light emitted by the light unit.

6. The cervix examination set as claimed in claim 4, wherein the light unit is capable of emitting white, green, blue, yellow, or purple light.

7. The cervix examination set as claimed in claim 5, wherein the light unit is capable of emitting white, green, blue, yellow, or purple light.

8. The cervix examination set as claimed in claim 4, wherein the light unit is a light emitting diode.

9. The cervix examination set as claimed in claim 4, wherein the inspection apparatus comprises a flexible tube, the light unit being located at an end of the flexible tube.

10. The cervix examination set as claimed in claim 4, wherein the inspection apparatus further comprises a switch, and the switch is used for controlling the control unit.

11. The cervix examination set as claimed in claim 4, wherein the closed portion of the container is in a slender form.

12. The cervix examination set as claimed in claim 4, wherein the closed portion of the container is L-shaped.

13. The cervix examination set as claimed in claim 4, wherein the inspection apparatus further comprises:
an image sensing unit for sensing images.

14. The cervix examination set as claimed in claim 13, wherein the image sensing unit is a charge coupled device or a complementary metal oxide semiconductor.

15. The cervix examination set as claimed in claim 13, wherein the inspection apparatus further comprises a wireless transmitter for transmitting image data generated by the image sensing unit.

16. The cervix examination set as claimed in claim 13, wherein the inspection apparatus further comprises a memory unit for saving image data generated by the image sensing unit.

17. The cervix examination set as claimed in claim 13, wherein the inspection apparatus further comprises a display unit for displaying image data generated by the image sensing unit.

18. The cervix examination set as claimed in claim 17, wherein the display unit is a liquid crystal display.

19. The cervix examination set as claimed in claim 13, wherein the inspection apparatus further comprises a wireless transmitter, a memory unit, and a display unit, wherein the wireless transmitter is for transmitting image data generated by the image sensing unit, the memory unit is for saving image data generate by the image sensing unit, and the display unit is for displaying image data generated by the image sensing unit.
